(19) United States
(12) Patent Application Publication
(10) Pub. No.: US 2008/0227831 A1
(43) Pub. Date: Sep. 18, 2008

(54) FOOD EXTRACTS FOR TREATMENT OF LIPOPROTEIN ABNORMALITIES AND SKIN DISEASES AND SKIN DISORDERS

(75) Inventors: Robert Bender, Ottawa (CA); Jerzy Gebicki, Lodz (PL)

Correspondence Address: LAHIVE & COCKFIELD, LLP;FLOOR 30, SUITE 3000, ONE POST OFFICE SQUARE, BOSTON, MA, 02109, US

(73) Assignee: Dermena, Ottawa (CA)

(21) Appl. No.: 11/874,627

(22) Filed: Oct. 18, 2007

Related U.S. Application Data
(60) Provisional application No. 60/852,586, filed on Oct. 18, 2006.

(51) Int. Cl. A61K 31/455; A61P 3/00; A61P 9/10; A61P 17/00; C07D 213/82

(52) U.S. Cl. 514/355; 546/316

(57) ABSTRACT

The present invention is directed to N-methylnicotinamide containing food extracts, and their use in treating lipoprotein abnormalities and skin diseases and disorders.

### RELATED APPLICATION

This application claims priority to U.S. Provisional Application No. 60/852,586, Attorney Docket No. PRI-009-1, filed Oct. 18, 2006, titled “Food Extracts for Treatment of Lipoprotein Abnormalities and Skin Diseases and Disorders,” which is incorporated herein by reference in its entirety. Additionally, the contents of any patents, patent applications, and references cited throughout this specification are hereby incorporated by reference in their entireties.

### BACKGROUND OF THE INVENTION

It has been clear for several decades that high total cholesterol, high triglycerides, low high-density lipoprotein cholesterol, normal to elevated low-density lipoprotein cholesterol, or small low-density lipoprotein particles are related to a variety of diseases, conditions and disorders.

The evidence linking elevated serum cholesterol to coronary heart disease is overwhelming (Badimon et al., Circulation, 86 Suppl. III, 1992, 86-94). Circulating cholesterol is carried by plasma lipoproteins, which are complex particles of lipid and protein that transport lipids in the blood. Low density lipoprotein (LDL) and high density lipoprotein (HDL) are the major cholesterol-carrier proteins. Id. LDL is believed to be responsible for the delivery of cholesterol from the liver, where it is synthesized or obtained from dietary sources, to extrahepatic tissues in the body. The term “reverse cholesterol transport” describes the transport of cholesterol from extrahepatic tissues to the liver, where it is catabolized and eliminated. It is believed that plasma HDL particles play a major role in the reverse transport process, acting as scavengers of tissue cholesterol. Id. HDL is also responsible for the removal non-cholesterol lipid, oxidized cholesterol and other oxidized products from the bloodstream.

Atherosclerosis, for example, is a slowly progressive disease characterized by the accumulation of cholesterol within the arterial wall. Compelling evidence supports the belief that lipids deposited in atherosclerotic lesions are derived primarily from plasma apolipoprotein B (apo B)-containing lipoproteins, which include chylomicrons, CLDL, IDL and LDL. See Badimon et al., 1992, Circulation 86:(Suppl. III)86-94. The apo B-containing lipoprotein, and in particular LDL, has popularly become known as the “bad” cholesterol. In contrast, HDL serum levels correlate inversely with coronary heart disease. Indeed, high serum levels of HDL is regarded as a negative risk factor. It is hypothesized that a high level of plasma HDL is not only protective against coronary artery disease, but may actually induce regression of atherosclerotic plaque. See Dansky and Fisher, 1999, Circulation 100: 1762-3. Thus, HDL has popularly become known as the “good” cholesterol.

Further, dyslipidemia is caused by various factors including, but not limited to, high total cholesterol, high triglycerides, low high-density lipoprotein cholesterol, normal to elevated low-density lipoprotein cholesterol, or small low-density lipoprotein particles.

Furthermore, both women and men are constantly seeking ways to maintain a youthful appearance for as long as possible and, consequently, seek to attenuate the signs of skin aging. The first visible signs of aging are usually found on the skin: dryness, fine lines and wrinkles, age spots, red blotches, and sagging and flaccid skin. Dullness and loss of hair are also well-known symptoms. As the skin ages, there is a reduction in protein synthesis, an increase in proteolysis and a general disruption of the skin barrier, connective tissue and cohesion.

Numerous skin or hair care products are available to consumers for treatment or prevention of these skin conditions that are caused by various external sources of stress, including, for example, atmospheric pollution, mechanical stress, contact with household and other chemicals, as well as sun exposure.

Also, men and women can develop diseases and disorders of the skin that can affect quality of life to a far greater extent than a sign of skin aging. Such diseases and disorders include, but are not limited to, burns, scalds and skin wounds.

Many compounds have been described as being useful for improving skin appearance and physiology, including reducing fine lines, wrinkles and other symptoms associated with aged or photodamaged skin. Also, many compounds and compositions are available for the treatment of more serious skin diseases and disorders.

Thus, there is a continued need to find new therapeutic agents to treat lipoprotein abnormalities. Accordingly, there is a great need to develop compounds and pharmaceutical compositions that will raise HDL levels, lower LDL levels, and/or lower triglyceride levels in a subject. Also, a continued need exists to find new therapeutic agents to counteract anti-aging effects as well as treat human skin diseases and disorders.

### SUMMARY OF THE INVENTION

There remains a need for new treatments and therapies that will raise HDL levels, lower LDL levels, and/or lower triglyceride levels in a subject. Also, a continued need exists to find new therapeutic agents to counteract anti-aging effects as well as treat human skin diseases and disorders. In one aspect, the invention provides a wakame extract that is enriched with MNA.

In one aspect, the invention provides a method of treating a lipoprotein abnormality in a subject in need thereof by administering to the subject a food extract containing N-methylnicotinamide. In one embodiment, the food extract is a seaweed extract. In another embodiment, the seaweed is wakame. In another embodiment, the lipoprotein abnormality is atherosclerosis. In still another embodiment, the food extract is administered orally. In another embodiment, the food extract is mixed with food stuff; wherein the food stuff is selected from the group consisting of cereals, bread, drinks, health bars, juices, concentrates, canned food, ice cream, water, staple goods, such as corn, barley, wheat and oat in any form, and/or taste maskers such as sugar or ascorbic acid. In another embodiment, the invention provides a method of treating a lipoprotein abnormality in a subject in need thereof by topically administering to the subject a food extract containing N-methylnicotinamide.

In another embodiment, the daily dose of food extract is between 1.0 mg/kg and 1000 mg/kg. In still another embodiment, the daily dose of food extract is between 5.0 mg/kg and 500 mg/kg. In yet another embodiment, the daily dose of food extract is between 6.0 mg/kg and 100 mg/kg.

In one embodiment, the subject to be treated is a mammal. In another embodiment, the mammal is human.

In one embodiment, the lipoprotein abnormality to be treated by the invention is a disease or disorder associated with the development and progress of atherosclerosis, hyperlipidaemias, angina pectoris or cardiac risk. In another embodiment, the disease or disorder associated with the development and progress of atherosclerosis is hypertension, dyslipidaemias, diabetes or obesity. In yet another embodiment, the treatment of atherosclerosis slows the progression of atherosclerotic plaques. In another embodiment, the progression of atherosclerotic plaques is slowed in coronary arteries.

In still another embodiment, the progression of atherosclerotic plaques is slowed in carotid arteries. In yet another embodiment, the progression of atherosclerotic plaques is slowed in the peripheral arterial system. In still another embodiment, the treatment of atherosclerosis causes the regression of atherosclerotic plaques. In yet another embodiment, the regression of atherosclerotic plaques occurs in coronary arteries. In still another embodiment, the lipoprotein abnormality is associated with hypertension, cerebral vasospasm, coronary vasospasm, bronchial asthma, preterm labor, erectile dysfunction, glaucoma, vascular smooth muscle cell proliferation, myocardial hypertrophy, malignoma, ischemia-induced injury, reperfusion-induced injury, endothelial dysfunction, Crohn's Disease and colitis, neurite outgrowth, Raynaud's Disease, angina, Alzheimer's disease or benign prostatic hyperplasia.

In another embodiment, the lipoprotein abnormality is associated with erectile dysfunction, reperfusion, ischemia, or vasospasm. In still another embodiment, the lipoprotein abnormality is associated with dementia or cancer. In one embodiment, the cancer is selected from the group consisting of prostate, skin, lung, colon, bladder, uterus and kidney cancer.

In another embodiment, the lipoprotein abnormality is associated with cardiovascular disease, peripheral vascular disease, dyslipidemia, dyslipoproteinemia, restenosis, a disorder of glucose metabolism, Alzheimer's Disease, Syndrome X, a peroxisome proliferator activated receptor-associated disorder, septicemia, a thrombotic disorder, obesity, pancreatitis, hypertension, renal disease, inflammation, inflammatory muscle diseases, such as polymylagia rheumatica, polymyositis, fibrositis, gastrointestinal disease, irritable bowel syndrome, inflammatory bowel disease, inflammatory disorders, impotence, arthritis, osteoporosis, soft tissue rheumatism, autoimmune disease, scleroderma, ankylosing spondylitis, gout, pseudogout, non-insulin dependent diabetes mellitus, septic shock, polycystic ovarian disease, hyperlipidemias, lipoprotein lipase deficiencies, lipoprotein abnormalities associated with diabetes, lipoprotein abnormalities associated with obesity, and lipoprotein abnormalities associated with Alzheimer's Disease.

In another aspect, the invention provides a method of treating atherosclerosis in a subject in need thereof by administering to the subject a food extract containing N-methylnicotinamide. In still another aspect, the invention provides a method of lowering LDL-cholesterol levels in a subject in need thereof by administering to the subject a food extract containing N-methylnicotinamide. In yet another aspect, the invention provides a method of raising HDL-cholesterol levels in a subject in need thereof by administering to the subject a food extract containing N-methylnicotinamide. In a particular embodiment, the food extract is a seaweed extract. In still another embodiment, the seaweed is wakame.

In one embodiment, the food extract is administered topically. In another embodiment, the food extract is administered orally. In another embodiment, the food extract is mixed with food stuff; wherein the food stuff is selected from the group consisting of cereals, bread, drinks, health bars, juices, concentrates, canned food, ice cream, water, staple goods, such as corn, barley, wheat and oat in any form, or taste maskers such as sugar or ascorbic acid.

In still another embodiment, the food extract is co-administered with a statin. In one embodiment, the statin is mevastatin, lovastatin, simvastatin, pravastatin, fluvastatin, pitavastatin, atorvastatin, cerivastatin, rosuvastatin, pentostatin, or nystatin, or a pharmaceutically acceptable salt, solvate, clathrate, polymorph, prodrug, or pharmacologically active metabolite thereof. In one embodiment, the statin and food extract are administered sequentially to the subject. In another embodiment, the statin and food extract are administered orally, nasally, rectally, intravaginally, parenterally, buccally, sublingually or topically.

In another embodiment, the statin and food extract are formulated using one or more pharmaceutically acceptable excipients chosen from starch, sugar, cellulose, diluent, granulating agent, lubricant, binder, disintegrating agent, wetting agent, emulsifier, coloring agent, release agent, coating agent, sweetening agent, flavoring agent, perfuming agent, preservative, antioxidant, plasticizer, gelling agent, thickener, hardener, setting agent, suspending agent, surfactant, humectant, carrier, stabilizer, or a combination thereof.

In another embodiment, the food extract further comprises a pharmaceutically acceptable carrier or excipient. In another embodiment, the food extract is administered with one or more pharmaceutically acceptable carriers, diluents or excipients. In another embodiment, the food extract is in tablet form. In another embodiment, the food extract is in capsule form. In still another embodiment, the food extract is in controlled release or sustained release form.

In another aspect, the invention provides a method of treating skin diseases and disorders in a subject in need thereof by administering to the subject a topical composition comprising wakame extract. In one embodiment, the wakame extract is enriched in MNA. In one embodiment, the skin diseases or disorders are selected from the group consisting of sunburn, burns, scalds, skin wounds, wrinkles, oxidative damage in the skin and UV-induced skin damage.

In one embodiment, the composition is applied on a daily basis. In another embodiment, the composition is administered for at least two weeks. In still another embodiment, the composition is administered for at least one month. In yet another embodiment, the composition is administered for at least two months. In another embodiment, the composition is administered for at least three months.

In another embodiment, the topical composition is formulated in a cream, a balm, an ointment, a liposome formulation, aqueous solution or a gel. In still another embodiment, the topical composition contains an additional component selected from the group consisting of water, glycerine, petrolatum, mineral oil micro-crystalline waxes, paraffins, ozokerite, polyethylene, polybutene, polydecene and perhydrosqualene, dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and any combinations thereof. In yet another embodiment, the topical composition is administered with the assistance of ultrasound radiation.

In another aspect, the invention provides a topical composition comprising a wakame extract enriched with MNA.

### DETAILED DESCRIPTION OF THE INVENTION

It is well known that nicotinic acid (NAc) in high doses possesses important properties in the correction of lipoprotein profile (i.e., the treatment of lipoprotein abnormalities), mostly by reducing triglyceride (TG) and elevating HDL levels. The main disadvantage of nicotinic acid therapy is associated with its side effects. Very frequently, cutaneous vasolidation and flushing are observed.

Studies have demonstrated that a pyridinium salt, namely, N-methylnicotinamide (MNA), is a molecule that can be used for the treatment of lipoprotein abnormalities (see, e.g., U.S. patent application Ser. No. 11/484,892, incorporated herein by reference). MNA is bound as a cationic molecule to Sepharose immobilized heparin (see, e.g., International Application No. PCT/EP2005/050057, and U.S. patent application Ser. No. 11/870,307, whish is incorporated herein by reference). It was found that MNA releases PGI₂ and it is cytoprotective to various cell lines. In addition, MNA is chemically very stable, non-toxic and very well tolerated.

The use of 1-alkylnicotinamide salts for treatment of a wide variety of skin diseases and disorders is described in EP Patent No. 1 147 086 (incorporated herein by reference). MNA is naturally found in food products like seaweed, e.g. wakame (Undaria pinnatifida) (see Taguchi et al., Vitamins (Japan) 1986, 60(11), 537-46). It has been demonstrated that foods containing MNA, in particular wakame, lead to decreases in serum and liver triglycerol levels in rats (see Murata et al., J Nutr. 1999 29 146-51 and Murata et al., J Nutr. 2002 132, 742-747).

Furthermore, wakame is a common additive in a variety of cosmetics due to its moisturising and anti-aging properties. As such, an MNA-containing food extract, e.g., MNA-containing wakame extract, may be used as a cosmetic additive.

The present invention is directed to food extracts containing MNA, e.g. seaweed extract, and their use in treating disorders, such as lipoprotein abnormalities. The present invention is also directed toward food extracts containing MNA, e.g. seaweed extract, and their use for the treatment of skin diseases and disorders, including, but not limited to, sunburn, burn, scalds, skin wounds, wrinkles, oxidative damage to the skin, UV-induced skin damage, and other effects of aging. In particular, specific embodiments of the invention are described herein as exemplary embodiments and are not intended to be limiting.

### DEFINITIONS

These and other embodiments of the invention will be described with reference to following definitions that, for convenience, are collected here.

The language “extract” is used to refer to any substance, liquid or solid, that is extracted from an MNA-containing food (e.g., wakame) with a higher concentration of MNA than in the original food source. For example, the MNA content in wakame is approximately 3.2 mg/100 g (see, e.g., Taguchi et al., Vitamins (Japan) 1986, 60(11), 537-46, which is incorporated herein by reference). A “wakame extract” is any substance, liquid or solid, that is extracted from wakame that has a concentration of MNA that is greater than 3.2 mg/100 g. Such a substance is said to be “enriched” in MNA. Additionally, the term “extract” refers to either the MNA that is extracted from food (e.g., actual MNA extracted from wakame seaweed), or a substance that is extracted from food that contains both MNA and other natural products that are derived from the food during the extraction process (e.g., MNA with magnesium and other trace minerals that are found in wakame). The term “extract” can also refer to the MNA that is extracted from food, along with any additional solvent, such as water, that was used to extract the MNA from the food. Furthermore, the wakame extract may be further combined with MNA, e.g., pure MNA extract, synthesized MNA or MNA purchased from a commercial supplier.

In a particular embodiment, the extract of the invention is a wakame extract that has a range of 4 mg-100 mg of MNA per 100 total grams, e.g., 4 mg-99 mg of MNA per 100 total grams, e.g., 4 mg-15 mg of MNA per 100 total grams, 15.1 mg-30 mg of MNA per 100 total grams, 30.1 mg-45 mg of MNA per 100 total grams, 45.1 mg-60 mg of MNA per 100 total grams; 60.1 mg-75 mg of MNA per 100 total grams, 75.1 mg-90 mg of MNA per 100 total grams, or 90.1 mg-99 mg of MNA per 100 total grams, or 90.1 mg-100 mg of MNA per 100 total grams.

In one embodiment, the “extract” is a water-based liquid that is enriched in MNA. Moreover, the term “extract” includes any material resulting from crushing the food (e.g., seaweed) and mixing with water or other ingredients; chopping, grinding, mincing, or forming a paste of the food, processing the food into a dry powder, extruding, fermenting, or any other process by which the MNA remains in the extract. Examples of food extracts of the invention include seaweed extract. In a preferred embodiment, the seaweed extract is wakame extract. In another embodiment, the wakame extract is a powder.

“Seaweed” includes, but is not limited to, such plant orders as: (1) Laminariaceae; (2) Fucaceae; and (3) Gigartinaceae. Ascophyllum nodosum is the most widely used form of seaweed utilized in agriculture and belongs to the order Fucaceae. Other important genus groups include Laminaria, Durvillea, Macrocystis, Chondrus, and Ecklonia. A preferred form of seaweed is wakame.

The term “cosmetic” or “cosmetic composition” or “cosmetic product” when used herein means any cosmetic product that can be directly applied to keratinous surfaces such as skin, hair, or nails, including, without limitation, lipstick, mascara, rouge, foundation, blush, eyeliner, lipliner, lip gloss, facial or body powder, sunscreens and blocks, nail polish, mousse, sprays, styling gels, nail conditioner, whether in the form of creams, lotions, gels, ointments, emulsions, colloids, solutions, suspensions, compacts, solids, pencils, spray-on formulations, brush-on formulations and the like. Personal care products that are described by the terms “cosmetic” or “cosmetic composition” or “cosmetic product” include, without limitation, bath and shower gels, shampoos, conditioners, cream rinses, hair dyes and coloring products, leave-on conditioners, sunscreens and sunblocks, lip balms, skin conditioners, hair sprays, soaps, body scrubs, exfoliants, astringents, depilatories and permanent waving solutions, antidandruff formulations, antisweat and antiperspirant compositions, shaving, preshaving and after shaving products, moisturizers, cold creams, deodorants, cleansers, skin gels, rinses, whether in solid, powder, liquid, cream, gel, ointment, lotion, emulsions, colloids, solutions, suspensions, or other form.

The term “keratinous surface” means bodily surfaces such as skin, hair, or nails.

The language “lipoprotein abnormality,” as used herein, describes diseases and disorders that may be treated or prevented (or a symptom of such disease or disorder that may be reduced) by the composition of the invention. In particular, a lipoprotein abnormality is caused by either high total cholesterol, high triglycerides, low high-density lipoprotein cholesterol, normal to elevated low-density lipoprotein cholesterol, or small low-density lipoprotein particles in a subject, or any combination thereof. These factors have been shown to play a role in a variety of diseases and disorders, including, but not limited to, a disorder associated with the development and progress of atherosclerosis (e.g., hypertension, dyslipidaemias, diabetes or obesity), hyperlipidaemias, angina pectoris or cardiac risk, cerebral vasospasm, coronary vasospasm, bronchial asthma, preterm labor, vascular smooth muscle cell proliferation, myocardial hypertrophy, malignoma, ischemia/reperfusion-induced injury, endothelial dysfunction, Crohn's Disease, colitis, neurite outgrowth, Raynaud's Disease, angina, Alzheimer's disease, benign prostatic hyperplasia, reperfusion/ischemia (e.g., stroke), vasospasm (e.g., cerebral vasospasm or coronary vasospasm), dementia and cancer (e.g., prostate, skin, lung, colon, bladder, uterus and kidney cancer).

Lipoprotein abnormalities also include cardiovascular disease, peripheral vascular disease, dyslipoproteinemia, restenosis, disorders of glucose metabolism, Syndrome X, a peroxisome proliferator activated receptor-associated disorder, septicemia, a thrombotic disorder, pancreatitis, renal disease, inflammation, inflammatory muscle diseases (e.g., polymylagia rheumatica, polymyositis, or fibrositis), impotence, gastrointestinal disease, irritable bowel syndrome, inflammatory bowel disease, inflammatory disorders, asthma, vasculitis, ulcerative colitis, Kawasaki disease, Wegener's granulomatosis, multiple sclerosis, autoimmune chronic hepatitis, arthritis, rheumatoid arthritis, juvenile rheumatoid arthritis, osteoarthritis, osteoporosis, soft tissue rheumatism, tendonitis, bursitis, autoimmune disease, scleroderma, ankylosing spondylitis, gout, pseudogout, non-insulin dependent diabetes mellitus (NIDDM), septic shock, polycystic ovarian disease, hyperlipidemias, familial combined hyperlipidemia, lipoprotein lipase deficiencies, hypoalphalipoproteinemia, lipoprotein abnormalities associated with diabetes, lipoprotein abnormalities associated with obesity, and lipoprotein abnormalities associated with Alzheimer's Disease.

Lipoprotein abnormalities also include diseases and disorders associated with dysfunction of the vascular endothelium, oxidative stress, insufficient production of endothelial prostacyclin PGI₂, low HDL levels, and/or high triglyceride levels.

In a particular embodiment, the lipoprotein abnormality is an acute cardiovascular event associated with atherosclerosis, in particular sudden cardiac death, acute coronary syndrome (including unstable coronary artery disease, and myocardial infarct), the necessity of coronary angioplasty, coronary-aortal by-pass surgery (CABG), any type of surgery with extracorporeal circulation, ischemic stroke, or peripheral circulation revascularization.

In another particular embodiment, the lipoprotein abnormality is atherosclerosis in patients with chronic coronary disease, ischemic cerebrovascular episode or artherosclerosis of the extremities, including obliterans.

In another particular embodiment, the lipoprotein abnormality is a condition or disease selected from the group of risk factors for atherosclerosis, comprising the following: hypercholesterolemia, arterial hypertension, smoking, hyperhomocysteinaemia, insulin resistance, menopause, aging, mental stress, infections, inflammatory states, including periodontal diseases, allograft vasculopathy and nitrate tolerance.

In another particular embodiment, the lipoprotein abnormality is dyslipidemia, in particular hypercholesterolemia or hypertriglyceridemia.

In another particular embodiment, the lipoprotein abnormality is thrombosis that is not related directly with atherosclerosis, in particular thrombosis associated with implantation of metallic vascular prostheses (stents), by-pass surgery hemodialysis or venous disease.

In another particular embodiment, the lipoprotein abnormality is selected from the following group: chronic heart failure, pulmonary hypertension, diabetic complications, such as diabetic retinopathy and diabetic neuropathy, nephrotic syndrome, chronic renal failure, adults respiratory distress syndrome, cystic fibrosis, chronic obstructive pulmonary disease, erectile dysfunction, sleep apnea, systemic lupus erythematosus, sickle cell anemia, non-specific inflammatory bowel diseases, gastric or duodenal ulcers, glaucoma, chronic liver disease, primary amyloidosis, and neurodegenerative diseases.

In a particular embodiment, lipoprotein abnormalities can be treated by raising HDL levels in a subject, decreasing LDL levels in a subject, lowering triglycerides in a subject, and/or lowering total cholesterol in a subject by administering to the subject in need thereof the food extracts of the invention, which contain MNA.

The language “skin diseases and disorders,” as used herein, describes diseases and disorders that may be treated or prevented (or a symptom of such disease or disorder that may be reduced) by the compounds of the invention. For example, skin diseases and disorders include, but are not limited to, skin diseases and disorders in which oedema, erythema, cutaneous eruption, dilation of superficial blood vessels and desquamation are manifested (including when accompanied by pruritus and burning sensation), as well as in cases of intensified seborrhoea. Skin diseases and disorders also include, but are not limited to, crural ulceration, acne juvenile, acne rosacea, psoriasis, atopic dermatitis and vitiligo. Skin diseases and disorders to be treated by the compositions of the invention also include, but are not limited to, hair loss, especially alopecia greata, androgenic alopecia, and alopecia caused as a side effect of chemotherapy or radiotherapy. Skin diseases and disorders to be treated by the compositions of the invention also include, but are not limited to, burns and scalds (particularly first and first/second degree burns and scalds) and in wound healing, as well as in treating sunburn.

In a particular embodiment, the “skin diseases and disorders” to be treated by the compositions of the invention are selected from the group consisting of sunburn, burns, scalds, skin wounds, wrinkles, oxidative damage in the skin, UV-induced skin damage and any other symptom of the aging process.

The term “treatment” or “treating,” as used herein, is defined as the application or administration of a therapeutic agent, i.e., an MNA-containing food extract, e.g., an MNA-containing wakame extract, to a subject, or application or administration of a therapeutic agent to an isolated tissue or cell line from a subject (e.g., for diagnosis or ex vivo applications), who has a lipoprotein abnormality, a symptom of a lipoprotein abnormality or a predisposition toward a lipoprotein abnormality, with the purpose to cure, heal, alleviate, relieve, alter, remedy, ameliorate, improve or affect the lipoprotein abnormality, the symptoms of the lipoprotein abnormality or the lipoprotein abnormality. Such treatments may be specifically tailored or modified, based on knowledge obtained from the field of pharmacogenomics.

The term “treatment” or “treating” also refers to administration of a cosmetic agent, i.e., a cosmetic agent containing an MNA-containing food extract, e.g., an MNA-containing wakame extract, to a subject, who has a skin disease or disorder, a symptom of a skin disease or disorder, or a predisposition toward a skin disease or disorder, with the purpose to cure, heal, alleviate, relieve, alter, remedy, ameliorate, improve or affect the skin disease or disorder.

The term “subject” includes living organisms in which lipoprotein abnormalities can occur, or which are susceptible to lipoprotein abnormalities. The term “subject” includes animals (e.g., mammals, e.g., cats, dogs, horses, pigs, cows, goats, sheep, rodents, e.g., mice or rats, rabbits, squirrels, bears, primates (e.g., chimpanzees, monkeys, gorillas, and humans)), as well as chickens, ducks, geese, and transgenic species thereof; and cells, e.g., immortalized or nonimmortalized cells, derived therefrom.

Administration of the food extracts of the present invention to a subject to be treated can be carried out using known procedures, at dosages and for periods of time effective to inhibit lipoprotein abnormalities in the subject. An effective amount of the food extracts necessary to achieve a therapeutic effect may vary according to factors such as the state of the disease or disorder in the subject, the age, sex, and weight of the subject, and the ability of the therapeutic compound to inhibit the lipoprotein abnormalities or in the subject. Dosage regimens can be adjusted to provide the optimum therapeutic response. For example, several divided doses may be administered daily or the dose may be proportionally reduced as indicated by the exigencies of the therapeutic situation. A non-limiting example of an effective dose range for the food extracts of the invention (e.g., amount of MNA in the food extract) is between 1 and 500 mg/kg of body weight/per day. One of ordinary skill in the art would be able to study the relevant factors and make the determination regarding the effective amount of the therapeutic compound without undue experimentation.

Actual dosage levels of MNA in the food extracts of this invention may be varied so as to obtain an amount of the active ingredient which is effective to achieve the desired therapeutic response for a particular patient, composition, and mode of administration, without being toxic to the patient. The food extract, e.g., wakame extract, may be administered with one or more glycoaminoglycans (GAGs) to control the release dynamics of the MNA contained in the wakame. Without being bound by theory, MNA can effectively bind GAGs; this binding may be due to formation of complexes based on electrostatic interactions. For example, such binding (and eventual occurrence of a non-binding event) can result in a timed release of the MNA. Suitable GAGs for administration with a wakame extract include, but are not limited to, heparin, heparin sulfate, keratan sulfate, dermatin, dermatin sulfate, heparin-hyaluronic acid, chondroitin, chondroitin sulfate (e.g., chondroitin 6-sulfate and chondroitin 4-sulfate), chitin, chitosan, acetyl-glucosamine, hyaluronic acid, aggrecan, decorin, biglycan, fibromodulin or lumican, or combinations thereof. (See, e.g., U.S. patent application Ser. No. 11/870,307, which is incorporated herein by reference.)

In particular, the selected dosage level will depend upon a variety of factors including the activity of the particular compound of the present invention employed, the time of administration, the rate of excretion of the particular compound being employed, the duration of the treatment, other drugs, compounds or materials used in combination with the particular compound employed, the age, sex, weight, condition, general health and prior medical history of the patient being treated, and like factors well known in the medical arts.

A medical doctor, e.g., physician or veterinarian, having ordinary skill in the art can readily determine and prescribe the effective amount of the pharmaceutical composition required. For example, the physician or veterinarian could start doses of the food extract of the invention at levels lower than that required in order to achieve the desired therapeutic effect and gradually increase the dosage until the desired effect is achieved.

The regimen of administration can affect what constitutes an effective amount. The therapeutic formulations can be administered to the subject either prior to or after the onset of a lipoprotein abnormality. Further, several divided dosages, as well as staggered dosages, can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

In particular embodiments, it is especially advantageous to formulate compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a lipoprotein abnormality in subjects.

### Food Extracts

The food extracts of the invention can be acquired using extraction techniques well-known to one skilled in the art. Examples of procedures that may be used to produce the extracts of the invention, particularly seaweed extracts, can be found, for example, in U.S. Pat. Nos. 7,074,440; 6,342,342; 6,689,376, 6,656,229; 6,528,106; 6,391,331; 3,948,881 and US Patent Application Nos. 20050196410, 20060116333, 20050196410, 20060088627 and 20050129828, as well as Vitamins (Japan), 63(11), 537-546 (1986), all of which are incorporated herein by reference in their entirety. A procedure for the production of wakame extract is also provided herein in the Exemplification section.

In a preferred embodiment, the food extracts of the invention slow the progression of atherosclerotic plaques (e.g., progression of atherosclerotic plaques is slowed in coronary arteries, in carotid arteries, in the peripheral arterial system) or cause the regression of atherosclerotic plaques.

In a another preferred embodiment, the food extracts of the invention, e.g., wakame extract, raise HDL levels in a subject, decrease LDL levels in a subject, lower triglycerides in a subject, and/or lower total cholesterol in a subject.

In one embodiment, the invention provides a method of treating atherosclerosis in a subject in need thereof by administering to the subject wakame extract.

In one embodiment, the invention provides a method of lowering LDL-cholesterol levels in a subject in need thereof by administering to the subject wakame extract.

In one embodiment, the invention provides a method of raising HDL-cholesterol levels in a subject in need thereof by administering to the subject wakame extract.

Without being bound by theory, it is believed that the MNA that naturally occurs in the food extracts of the invention, particularly seaweed extracts, e.g., wakame extract, are effective in treating lipoprotein abnormalities for the following reasons: on the surface of the vascular endothelium, polyanionic molecules, such as glycosaminoglycans, are present and it would be expected that the molecules able to manifest some endothelial potential should be bound to vascular endothelium. MNA, which is positively charged, binds to the negatively charged glycosamionoglycans present on the vascular endothelium surface due to electrostatic interactions. This binding can result in manifestation of various endothelial effects, some of which can be positive from pharmacologic view point, for example release of NO and/or prostacyclin. Further, this activity can result in the treatment or prevention of lipoprotein abnormalities (which can be caused by, e.g., high total cholesterol, high triglycerides, low high-density lipoprotein cholesterol, normal to elevated low-density lipoprotein cholesterol, or small low-density lipoprotein particles in the subject).

The MNA-containing food extracts of the invention, e.g., MNA-containing wakame extract, may be used as a cosmetic additive. The cosmetic utility of an MNA-containing food extracts, e.g., an MNA-containing wakame extract includes, but is not limited to, a moisturising cosmetic or anti-aging cosmetic. In a particular embodiment, wakame extract is added to a cosmetic that is a gel, ointment or cream that has moisturizing and/or anti-aging properties.

In a preferred embodiment, the invention provides an anti-wrinkle cream effective for restoring firmness and tonicity to the skin of a subject, wherein the anti-wrinkle cream comprises wakame extract.

In another preferred embodiment, the invention provides a therapeutic effective for treating burns, scalds, and skin wounds in a subject, wherein the therapeutic comprises wakame extract.

Without being bound by theory, it is believed that the food extracts of the invention are effective in treating skin diseases and disorders (e.g., cuts and wounds) for the following reasons: 1-alkylnicotinamide salts, e.g., 1-methylnicotinamide salts (MNA) and the related pyridinium salts can effectively bind glycosaminoglycans; this binding may be due to formation of complexes based on electrostatic interactions. Such binding may facilitate MNA transport into the skin, which leads to the treatment of skin diseases and disorders, e.g., wrinkles. (MNA is a highly hydrophilic molecule with a solubility in water of over 600 g/L.)

Using the food extracts of the invention as a source of MNA, MNA for administration can be in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

Using the food extracts of the invention as a source of MNA, MNA for administration can be in the range of between about 0.0001 mg and about 25 mg. In some embodiments, a dose of a MNA used in compositions described herein is less than about 100 mg, or less than about 80 mg, or less than about 60 mg, or less than about 50 mg, or less than about 30 mg, or less than about 20 mg, or less than about 10 mg, or less than about 5 mg, or less than about 2 mg, or less than about 0.5 mg. Similarly, in some embodiments, a dose of a second compound (i.e., a statin) as described herein is less than about 1000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg.

### Combination Therapies

The food extracts, e.g., wakame extract, of the present invention can be intended to be useful, e.g., in the methods of present invention, in combination with one or more additional compounds useful for treating lipoprotein abnormalities. These additional compounds may comprise compounds of the present invention or compounds, e.g., commercially available compounds, known to treat, prevent, or reduce the symptoms of a lipoprotein abnormality.

In particular, the food extracts e.g., wakame extract, of the invention can be co-administered with statins. The term “statin,” where used in the specification and the appendant claims, is synonymous with the terms “3-hydroxy-3-methylglutary-1-Coenzyme A reductase inhibitor” and “HMG-CoA reductase inhibitor.” These three terms are used interchangeably in the art. As the synonyms suggest, statins are inhibitors of 3-hydroxy-3-methylglutaryl Coenzyme A reductase and, as such, are effective in lowering the level of blood plasma cholesterol. Statins and pharmaceutically acceptable salts thereof are particularly useful in lowering low-density lipoprotein cholesterol levels in mammals, and particularly in humans.

Statins suitable for use in the compositions and methods of the invention are also disclosed in U.S. Pat. Nos. 4,681,893; 5,273,995; 5,356,896; 5,354,772; 5,686,104; 5,969,156; and 6,126,971, each of which is incorporated herein in its entirety by reference. As some statins may exist in an inactive form, such as a lactone (e.g., simvastatin), the invention encompasses using the active form (e.g., b-hydroxy acid form) of them. See Physicians Desk Reference, 54^{th} Ed. (2000) pp. 1917-1920.

Statins include red rice extract, mevastatin, lovastatin, simvastatin, pravastatin, fluvastatin, pitavastatin, atorvastatin, cerivastatin, rosuvastatin, pentostatin or nystatin, or a pharmaceutically acceptable salt, solvate, clathrate, polymorph, prodrug, or pharmacologically active metabolite thereof.

Preferred statins are those agents which have been marketed, most preferred are pravastatin (e.g., Pravachol™), fluvastatin, simvastatin (e.g., Zocor™), lovastatin (e.g., Mevacor™), atorvastatin, or pitavastatin or a pharmaceutically acceptable salt thereof.

The statins have also recently been reported to have potential utility in the treatment of dementia (The Lancet, 2000: 356; 1627-1631) and various cancers, e.g., prostate, skin, lung colon, bladder, uterus and kidney (Arch. Intern. Med. 2000, 160: 2363-2368). These disorders, which are treated herein as “lipoprotein abnormalities,” can be treated by an MNA-containing food extract of the invention.

In some embodiments, a food extract, e.g., wakame extract, of the invention and a statin are included in a single composition, which is administered to a subject having a lipoprotein abnormality. In other embodiments, a food extract of the invention and a statin are administered separately to such a subject. The first and at least one second agent may either be co-administered to a subject (i.e., at the same time) or be administered sequentially (i.e., one after the other).

A combination of compounds described herein can either result in synergistic increase in effectiveness against a lipoprotein abnormality, relative to effectiveness following administration of each compound when used alone, or such an increase can be additive. Compositions described herein typically include lower dosages of each compound in a composition, thereby avoiding adverse interactions between compounds and/or harmful side effects, such as ones which have been reported for similar compounds. Furthermore, normal amounts of each compound when given in combination could provide for greater efficacy in subjects who are either unresponsive or minimally responsive to each compound when used alone.

For example, statins have been associated with some side-effects, including myalgias, muscle cramps, myositis, myopathy, and other gastrointestinal problems. The administration of the food extracts of the invention, e.g., seaweed extracts, in combination with a statin to a subject in need thereof may serve to counteract unwanted side-effects associated with statin use.

A synergistic effect can be calculated, for example, using suitable methods such as, for example, the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

In another embodiment, the food extracts, e.g., wakame extract, of the invention can be co-administered with one or more steroids. The term “steroids” is, according to the invention, intended to comprise all natural and synthetic steroid hormones, their analogs and derivatives thereof such as sulphate and fatty acid esters, their precursors, metabolites and their analogs, which may be steroidal or not steroidal in structure. The term “steroids” includes compounds having the basic cyclopentanoperhydrophenanthrene ring structure and which may contain various substituents and/or double bonds, e.g., a keto, hydroxy or acyloxy group in the 3-position; alkyl groups in any of 2-, 4-, 10-, 13-, 14- and 16-positions; a keto, ketal or ortho ester group at the 20-position; a keto group, or hydroxy and/or hydrocarbon or acyl (e.g. acetoxyacetyl) groups at the 17-position; a hydroxy or keto group at the 11- or 12-position, a hydroxy group at the 6-, 7- or 20-position, an esterified hydroxy group at the 21-position, a double bond at 5-position or the 1- and/or 4-position and a halogen atom such as fluorine or chlorine in the 11- or 6-position.

The term “steroids” also includes semisynthetic or synthetic polycyclic molecules, capable of binding to human membrane steroid receptors, their mixtures, precursors and metabolites. Examples of steroids include, but are not limited to, flunisolide, beclomethasone, triamcinolone, budesonide, fluticasone, mometasone, ciclesonide, rofleponide, ST-126, and dexamethasone.

In another embodiment, the food extracts, e.g., wakame extract, of the invention can be co-administered with one or more NSAIDs.

As used herein, the term “NSAID” includes, but is not limited to, those agents which inhibit cyclooxygenase, the enzyme responsible for the biosyntheses of the prostaglandins and certain autocoid inhibitors, including inhibitors of the various isoenzymes of cyclooxygenase (including, but not limited to, cyclooxygenase-1 and -2), such as the commercially available NSAIDs aceclofenac, acemetacin, acetaminophen, acetaminosalol, acetyl-salicylic acid, acetyl-salicylic-2-amino-4-picoline-acid, 5-aminoacetylsalicylic acid, alclofenac, aminoprofen, amfenac, ampyrone, ampiroxicam, anileridine, bendazac, benoxaprofen, bermoprofen, α-bisabolol, bromfenac, 5-bromosalicylic acid acetate, bromosaligenin, bucloxic acid, butibufen, carprofen, celecoxib, chromoglycate, cinmetacin, clindanac, clopirac, sodium diclofenac, diflunisal, ditazol, droxicam, enfenamic acid, etodolac, etofenamate, felbinac, fenbufen, fenclozic acid, fendosal, fenoprofen, fentiazac, fepradinol, flufenac, flufenamic acid, flunixin, flunoxaprofen, flurbiprofen, glutametacin, glycol salicylate, ibufenac, ibuprofen, ibuproxam, indomethacin, indoprofen, isofezolac, isoxepac, isoxicam, ketoprofen, ketorolac, lornoxicam, loxoprofen, meclofenamic acid, mefenamic acid, meloxicam, mesalamine, metiazinic acid, mofezolac, montelukast, mycophenolic acid, nabumetone, naproxen, niflumic acid, nimesulide, olsalazine, oxaceprol, oxaprozin, oxyphenbutazone, paracetamol, parsalmide, perisoxal, phenyl-acethyl-salicylate, phenylbutazone, phenylsalicylate, pyrazolac, piroxicam, pirprofen, pranoprofen, protizinic acid, reserveratol, salacetamide, salicylamide, salicylamide-O-acetyl acid, salicylsulphuric acid, salicin, salicylamide, salsalate, sulindac, suprofen, suxibutazone, tamoxifen, tenoxicam, theophylline, tiaprofenic acid, tiaramide, ticlopridine, tinoridine, tolfenamic acid, tolmetin, tropesin, xenbucin, ximoprofen, zaltoprofen, zomepirac, tomoxiprol, zafirlukast and cyclosporine. Additional NSAID genera and particular NSAID compounds are disclosed in U.S. Pat. No. 6,297,260, incorporated entirely by reference (especially in the generic formulas of its claim 1 and the recitation of specific list of NSAIDs contained therein and in claim 3, and thiazulidene NSAIDs disclosed in International Patent Application WO 01/87890, incorporated herein by reference in its entirety). Preferred NSAIDs are indomethacin, flufenamic acid, flunixin and theophylline. Most preferred is indomethacin, voltaren and naprosyn. In certain embodiments, the NSAID subunit is neither acetyl salicylic acid or mycophenolic acid. Additionally, The Merck Manual, 16th Edition, Merck Research Laboratories (1990) pp 1308-1309, as well as The Pharmacological Basis of Therapeutics, 9th edition, Macmillan Publishing Co., 1996, pp 617-655, provide well known examples of NSAIDs.

### Formulations for Administration

The food extracts of the present invention are optionally formulated for oral, sublingual, subcutaneous, intravenous, transdermal or rectal administrations in dosages and in admixture with pharmaceutical excipients or vehicles including implantation or controlled-release devices. For example, the compound of the seaweed extract is optionally dispersed in a physiologically acceptable, non-toxic liquid vehicle, such as water.

Alternatively, the food extract can be given in tablet, capsule, powder, granules, coated tablet form, or mixed with various food stuffs such as: cereals, bread, drinks, health bars, juices, concentrates, canned food, ice cream, water, staple goods such as wheat, corn, barley, and oat in any form, processed or not, or taste maskers such as sugar or ascorbic acid, or other functional foods. The compound is made using conventional methods, and may be mixed with conventional pharmaceutical auxiliaries, such as binders, fillers, preservatives, tablet disintegrators, flow regulators, plasticizers, wetting agents, dispersants, emulsifiers, solvents, retarding agents and/or anti-oxidants. It is also optionally contained or formed into a complex with lipids in various formulations and molecular arrangements.

In another embodiment, the present invention is directed to a packaged pharmaceutical composition comprising a container holding a food extract of the invention; and instructions for using the compound to treat, prevent, or reduce one or more symptoms of one or more lipoprotein abnormalities in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing one or more lipoprotein abnormalities in a subject.

Another embodiment of the invention is a pharmaceutical composition comprising a therapeutically effective amount of a food extract containing MNA and a pharmaceutically acceptable carrier.

The language “therapeutically effective amount” describes the amount of food extract of the invention that is effective to treat one or more lipoprotein abnormalities in a subject.

The language “pharmaceutically acceptable carrier” includes a pharmaceutically acceptable material, composition or carrier, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin. In one embodiment, the pharmaceutically acceptable carrier is not DMSO alone.

The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For example, for oral administration the compounds can be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY™ film coating systems available from Colorcon, West Point, Pa. (e.g., OPADRY™ OY Type, OY-C Type, Organic Enteric OY-P Type, Aqueous Enteric OY-A Type, OY-PM Type and OPADRY™ White, 32K18400). Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid). As mentioned above, the food extracts of the invention may be mixed with food stuffs.

### Parenteral Administration

For parenteral administration, the compounds for use in the method of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

### Transmucosal Administration

Transmucosal administration is carried out using any type of formulation or dosage unit suitable for application to mucosal tissue. For example, the selected active agent can be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

### Transurethal Administration

With regard to transurethal administration, the formulation can comprise a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials. A transurethral permeation enhancer can be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone™ from Nelson Research & Development Co., Irvine, Calif.), SEPA™ (available from Macrochem Co., Lexington, Mass.), surfactants as discussed above, including, for example, Tergitol™, Nonoxynol-9™ and TWEEN-80™, and lower alkanols such as ethanol.

### Transrectal Administration

Transrectal dosage forms may include rectal suppositories, creams, ointments, and liquid formulations (enemas). The suppository, cream, ointment or liquid formulation for transrectal delivery comprises a therapeutically effective amount of the selected active agent and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration may be in the range of from about 10 minutes to about 6 hours, e.g., less than about 3 hours.

### Vaginal or Perivaginal Administration

Vaginal or perivaginal dosage forms may include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit can be fabricated to disintegrate rapidly or over a period of several hours. The time period for complete disintegration may be in the range of from about 10 minutes to about 6 hours, e.g., less than about 3 hours.

### Intranasal or Inhalation Administration

The active agents may also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators can be used. For liquid formulations, the active agent can be formulated into a solution, e.g., water or isotonic saline, buffered or unbuffered, or as a suspension. In certain embodiments, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers can also include nasal gels, creams, pastes or ointments with a viscosity of, e.g., from about 10 to about 6500 cps, or greater, depending on the desired sustained contact with the nasal mucosal surfaces. Such carrier viscous formulations may be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent.

Non-aerosol formulations for inhalation can take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations can contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation can also comprise dry powder formulations, particularly insufflations in which the powder has an average particle size of from about 0.1 μm to about 50 μm, e.g., from about 1 μm to about 25 μm.

### Topical Formulations

In a particularly preferred embodiment, the compositions of the invention are administered to a subject in a topical formulation. The topical compositions useful in the present invention may be made into a wide variety of product forms such as are known in the art. These include, but are not limited to, cosmetic and cosmetic compositions, as well as lotions, creams, gels, sticks, shampoos, soaps, sprays, ointments, pastes and mousses. These product forms may comprise several types of carriers including, but not limited to, solutions, aerosols, emulsions, gels, solids, and liposomes. Topical formulations are most suitably in the form of an ointment, gel, cream, shampoo, soap, spray, lotion or a solution.

Preferred is topical administration to the skin at the location of the principal manifestation of the skin disease or disorder, (e.g., wrinkle, burn or other skin wound).

The topical formulations of the present invention comprise a safe and effective amount of a dermatologically acceptable carrier within which the compositions of the invention are incorporated to enable the extracts of the invention to be delivered to the skin or other relevant site at an appropriate concentration. The carrier can thus act as a diluent, dispersant, solvent, or the like which ensures that the formulation can be applied to and distributed evenly over the selected target to provide an appropriate concentration of the composition of the invention.

Preferred topical formulations according to the present invention comprise about 90 to 99.95% of a pharmaceutical base carrier and about 0.005 to about 10% by weight of a composition of wakame extract. More preferably the topical formulation contains about 0.1 to about 5% by weight of a composition of wakame extract. Preferred pharmaceutical base carriers are an ointment, gel, or aqueous solution.

The carrier may contain one or more dermatologically acceptable solid, semi-solid or liquid fillers, diluents, solvents, extenders and the like. The carrier may be solid, semi-solid or liquid. Preferred carriers are substantially liquid. The carrier can itself be inert or it can possess dermatological benefits of its own. Concentrations of the carrier can vary with the carrier selected and the intended concentrations of the food extract (e.g., wakame extract) and the other optional components.

Suitable carriers for topical formulations include conventional or otherwise known carriers that are dermatologically acceptable. The carrier should also be physically and chemically compatible with the composition of the invention, and should not unduly impair stability, efficacy or other benefits associated with the formulations of the present invention. Preferred components of the formulations of the present invention should be capable of being comingled in a manner such that there is no interaction which would substantially reduce the efficacy of the formulation under ordinary use situations.

Preferred carriers contain a dermatologically acceptable, hydrophilic diluent. As used herein, “diluent” includes materials in which the composition of the invention can be dispersed, dissolved, or otherwise incorporated. Nonlimiting examples of hydrophilic diluents are water, organic hydrophilic diluents such as lower monovalent alcohols (e.g., C₁-C₄) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof. Water is a preferred diluent. The composition preferably comprises from about 60% to about 99.99% of the hydrophilic diluent

Solutions according to the subject invention typically include a dermatologically acceptable hydrophilic diluent. Solutions useful in the subject invention preferably contain from about 60% to about 99.99% of the hydrophilic diluent.

Aerosols according to the subject invention can be formed by adding a propellant to a solution such as described above. Exemplary propellants include chloro-fluorinated lower molecular weight hydrocarbons. Additional propellants that are useful herein are described in Sagarin, Cosmetics Science and Technology, 2nd Edition, Vol. 2, pp. 443-465 (1972), incorporated herein by reference. Aerosols are typically applied to the skin as a spray-on product

The topical compositions of the subject invention, including, but not limited to, lotions and creams, may comprise a dermatologically acceptable emollient. Such compositions preferably contain from about 2% to about 50% of the emollient. Emollients tend to lubricate the skin, increase the smoothness and suppleness of the skin, prevent or relieve dryness of the skin, and/or protect the skin. Emollients are typically water-immiscible, oily or waxy materials. A wide variety of suitable emollients are known and may be used herein. Sagarin, Cosmetics Science and Technology, 2nd Edition, Vol. 1, pp. 32-43 (1972), incorporated herein by reference, contains numerous examples of materials suitable as an emollient.

Lotions and creams according to the present invention generally comprise a solution carrier system and one or more emollients. Lotions typically comprise from about 1% to about 20%, preferably from about 5% to about 10%, of emollient; from about 50% to about 90%, preferably from about 60% to about 80%, water. A cream typically comprises from about 5% to about 50%, preferably from about 10% to about 20%, of emollient; and from about 45% to about 85%, preferably from about 50% to about 75%, water.

Ointments of the present invention may comprise a simple carrier base of animal or vegetable oils or semi-solid hydro-carbons (oleaginous); absorption ointment bases which absorb water to form emulsions; or water soluble carriers, e.g., a water soluble solution carrier. Ointments may further comprise a thickening agent, such as described in Sagarin, Cosmetics, Science and Technology, 2nd edition, Vol. 1, pp. 72-73 (1972), incorporated herein by reference, and/or an emollient. For example, an ointment may comprise from about 2% to about 10% of an emollient; and from about 0.1% to about 2% of a thickening agent.

Preferred ointments comprise Eucerine and glycerol; preferred gels comprise methylcellulose, glycerol and water, or comprise polyacrylic acid, polyethylene glycol, ethanol, triethanolamine, paraben and water; preferred solutions comprise aqueous solutions or solutions of ethyl alcohol or propylene glycol.

Carriers for topical formulations of the compositions of the invention may also include one or more vitamins, such as vitamin A or vitamin E.

Preferred carriers for topical formulations of the compositions of the invention include one or more of the following: polyglyceryl-2-dipolyhydroxystearate, dicaprylyl ether, cocoglycerides, cera alba, sorbitan sesquioleate, aluminium stearates, dicocoyl pentaerythrityl distearyl citrate, dicocoyl pentaerythrityl distearyl citrate, sorbitan sesquioleate, glycerin, ethylhexyl stearate, dicaprylyl carbonate, cocoglycerides, tocopheryl acetate, DMDM hydantoin, methylparaben, phenoxyethanol, propylparaben, vitamin A, vitamin E, and water.

### Transdermal Administration

The compounds of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

### Ultrasound Administration

The administration of the compositions of the invention to a subject may be ultrasound assisted. The term “ultrasound assisted,” as used herein, generally refers to the delivery of compositions of the invention (charged, uncharged, or mixtures thereof), through a body surface (such as skin, mucous membrane, or nails) wherein the delivery is at least partially induced or aided by the application of ultrasonic energy in the form(s) of high frequency sound waves and/or vibrations. As used herein, the term “ultrasound” or “ultrasound energy” is a broad term and is used in its ordinary sense and means, without limitation, mechanical energy transferred through pressure or compression waves with a frequency greater than about 20 KHz. In one embodiment, the waves of the ultrasound energy have a frequency between about 500 KHz and 20 MHz and in another embodiment between about 1 MHz and 3 MHz. In yet another embodiment, the waves of the ultrasound energy have a frequency of about 3 MHz. The term “ultrasound” includes diagnostic, therapeutic and focused ultrasound. Diagnostic ultrasound refers to an ultrasound energy source in a range up to about 100 mW/cm² (FDA recommendation). Therapeutic ultrasound refers to an ultrasound energy source in a range up to about 3-4 W/cm² (WHO recommendation).

Focused ultrasound (FUS) allows thermal energy to be delivered without an invasive probe (see Morocz et al. 1998 Journal of Magnetic Resonance Imaging Vol. 8, No. 1, pp. 136-142). Another form of focused ultrasound is high intensity focused ultrasound (HIFU) which is reviewed by Moussatov et al. in Ultrasonics 1998 Vol. 36, No. 8, pp. 893-900 and TranHuuHue et al. in Acustica, 1997, Vol. 83, No. 6, pp. 1103-1106.

In a particular embodiment, the compositions of the invention (e.g., wakame extract) are administered to a subject using “ultrasound assistance” from the U-Strip transdermal delivery system, A-wand antiseptic delivery system, and/or U-wand cosmetic delivery system as provided by Dermisonics (http://www.dermisonics.com/).

### Iontophoresis

The administration of the compositions of the invention to a subject may also be iontophoresis assisted. The term “iontophoresis,” as used herein, refers generally to the delivery of a therapeutic agent (charged, uncharged, or mixtures thereof) through a body surface (such as skin, mucous membrane, or nails) wherein the delivery is at least partially induced or aided by the application of an electric potential. As is known in the art, iontophoresis, an electrotransport process, involves the electrically induced transport of charged ions.

In many instances, more than one of the noted processes may be occurring simultaneously to different extents. Accordingly, the term “iontophoresis” is given herein its broadest possible interpretation, to include the electrically induced or enhanced transport of at least one charged or uncharged agent, or mixtures thereof (e.g., a wakame extract), regardless of the specific mechanism(s) by which the agent is actually being transported.

In typical transdermal iontophoresis system a low constant current, ranging from micro-Amps to several milli-Amps, is applied for prolonged periods of time ranging from minutes to days. Alternatively, low constant voltage, ranging from milli volts to several volts is applied for prolonged periods of time ranging from minutes to days. The target amperage or voltage may also be achieved by a slow ramping up of the applied electric condition. Alternatively, starting from the target amperage or voltage, the electrical conditions may also be ramped down over time. Alternatively, consecutive pulses using the above electrical conditions are applied during the total duration of iontophoresis. Collectively, the above electrical conditions are referred to herein as “iontophoresis energy”. The above conditions are different from the electrical conditions as applied in the field of electroporation and do not result in measurable pore formation through cell membrane.

Devices that deliver active substances using iontophoresis have been developed for many applications, most of which involve the delivery of pharmaceutical compounds through the subject's skin and into the circulatory system or other organs of a subject's body. Devices for the facilitation of the administration of the compositions of the invention to a subject using iontophoresis are known to those of skill in the art.

### Intrathecal Administration

One common system utilized for intrathecal administration is the APT

Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication can then be administered directly to cells in the spinal cord involved in conveying sensory and motor signals associated with lower urinary tract disorders.

### Intravesical Administration

The term intravesical administration is used herein in its conventional sense to mean delivery of a drug directly into the bladder. Suitable methods for intravesical administration can be found, for example, in U.S. Pat. Nos. 6,207,180 and 6,039,967.

### Additional Administration Forms

Additional dosage forms of this invention include dosage forms as described in U.S. Pat. No. 6,340,475, U.S. Pat. No. 6,488,962, U.S. Pat. No. 6,451,808, U.S. Pat. No. 5,972,389, U.S. Pat. No. 5,582,837, and U.S. Pat. No. 5,007,790. Additional dosage forms of this invention also include dosage forms as described in U.S. patent application Ser. No. 20030147952, U.S. patent application Ser. No. 20030104062, U.S. patent application Ser. No. 20030104053, U.S. patent application Ser. No. 20030044466, U.S. patent Application Ser. No. 20030039688, and U.S. patent application Ser. No. 20020051820. Additional dosage forms of this invention also include dosage forms as described in PCT Patent Application WO 03/35041, PCT Patent Application WO 03/35040, PCT Patent Application WO 03/35029, PCT Patent Application WO 03/35177, PCT Patent Application WO 03/35039, PCT Patent Application WO 02/96404, PCT Patent Application WO 02/32416, PCT Patent Application WO 01/97783, PCT Patent Application WO 01/56544, PCT Patent Application WO 01/32217, PCT Patent Application WO 98/55107, PCT Patent Application WO 98/11879, PCT Patent Application WO 97/47285, PCT Patent Application WO 93/18755, and PCT Patent Application WO 90/11757.

### Controlled Release Formulations and Drug Delivery Systems

In certain embodiments, the formulations of the present invention can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds. As such, the compounds for use the method of the invention can be administered in the form of microparticles for example, by injection or in the form of wafers or discs by implantation.

In a preferred embodiment of the invention, the MNA-containing food extract, are administered to a subject, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

### Dosing

The therapeutically effective amount or dose of a food extract of the present invention will depend on the age, sex and weight of the patient, the current medical condition of the patient and the nature of the lipoprotein abnormalities being treated. The skilled artisan will be able to determine appropriate dosages depending on these and other factors.

A suitable dose of a compound of the present invention (i.e., MNA found in a food extract) can be in the range of from about 0.001 mg to about 500 mg per day, such as from about 0.01 mg to about 100 mg, for example, from about 0.05 mg to about 50 mg, such as about 0.5 mg to about 25 mg per day. The dose can be administered in a single dosage or in multiple dosages, for example from 1 to 4 or more times per day. When multiple dosages are used, the amount of each dosage can be the same or different. For example a dose of 1 mg per day can be administered as two 0.5 mg doses, with about a 12 hour interval between doses.

It is understood that the amount of food extract dosed per day can be administered every day, every other day, every 2 days, every 3 days, every 4 days, every 5 days, etc. For example, with every other day administration, a 5 mg per day dose can be initiated on Monday with a first subsequent 5 mg per day dose administered on Wednesday, a second subsequent 5 mg per day dose administered on Friday, etc.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

### EQUIVALENTS

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including reaction times, reaction size/volume, and experimental reagents, such as solvents, catalysts, pressures, atmospheric conditions, e.g., nitrogen atmosphere, and reducing/oxidizing agents, etc., with art-recognized alternatives and using no more than routine experimentation, are within the scope of the present application.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values that fall within these ranges, as well as the upper or lower limits of a range of values, are also contemplated by the present application.

### INCORPORATION BY REFERENCE

The contents of all references, issued patents, and published patent applications cited throughout this application are hereby expressly incorporated by reference in their entireties. It should be understood that the use of any of the compounds described herein are within the scope of the present invention and are intended to be encompassed by the present invention and are expressly incorporated herein for all purposes.

### EXEMPLIFICATION OF THE INVENTION

The invention is further illustrated by the following examples, which should not be construed as further limiting.

### Example 1

### Preparation of Wakame Extract

Dried seaweed (100 g) was powdered in a coffee grinder and suspended in 500 mL of water—96% ethanol solution (2:1, v/v). The mixture was vigorously stirred for 2 hours at room temperature, then filtered through a paper filter. The filtrate was concentrated almost to dryness in a rotary evaporator (around 20 mmHg, temperature not exceeding 30° C.), the residue dissolved in 150 mL of water, stirred for 10 minutes at room temperature and filtered through a paper filter. Evaporation of water in a rotary evaporator followed by drying over P₂O₅ in a vacuum desiccator gave around 28 g of light-beige powder, which was hygroscopic.

1. A method of treating a lipoprotein abnormality in a subject in need thereof by administering to the subject a food extract containing N-methylnicotinamide.

2. The method of claim 1, wherein the food extract is a seaweed extract.

3. The method of claim 2, wherein the seaweed is wakame.

4. The method of claim 1, wherein with lipoprotein abnormality is atherosclerosis.

5. The method of claim 1 wherein the food extract is administered orally.

6. The method of claim 1 wherein the food extract is mixed with food stuff; wherein the food stuff is selected from the group consisting of cereals, bread, drinks, health bars, juices, concentrates, canned food, ice cream, water, staple goods, such as corn, barley, wheat and oat in any form, and taste maskers such as sugar or ascorbic acid.

7. The method of claim 1, wherein the food extract is administered topically.

8. The method of claim 1, wherein the lipoprotein abnormality is a disease or disorder associated with the development and progress of atherosclerosis, hyperlipidaemias, angina pectoris or cardiac risk.

9. The method of claim 8, wherein the disease or disorder associated with the development and progress of atherosclerosis is hypertension, dyslipidaemias, diabetes or obesity.

10. The method of claim 8, wherein said treatment of atherosclerosis slows the progression of atherosclerotic plaques.

11. A method of treating atherosclerosis in a subject in need thereof by administering to the subject a food extract containing N-methylnicotinamide.

12. The method of claim 11, wherein the food extract is a seaweed extract.

13. The method of claim 12, wherein the seaweed is wakame.

14. The method of claim 11, wherein the food extract is administered topically.

15. The method of claim 11, wherein the food extract is administered orally.

16. The method of claim 11, wherein the food extract is mixed with food stuff;
wherein the food stuff is selected from the group consisting of cereals, bread, drinks, health bars, juices, concentrates, canned food, ice cream, water, staple goods, such as corn, barley, wheat and oat in any form, or taste maskers such as sugar or ascorbic acid.

17. A method of treating skin diseases and disorders in a subject in need thereof by administering to the subject a topical composition comprising wakame extract.

18. The method of claim 17, wherein the skin diseases or disorders are selected from the group consisting of sunburn, burns, scalds, skin wounds, wrinkles, oxidative damage in the skin and UV-induced skin damage.

19. A wakame extract enriched with MNA.

20. The wakame extract of claim 19, wherein the extract is enriched with 4 mg-99 mg of MNA per 100 total grams.

21. The wakame extract of claim 19, wherein the extract is enriched with 45.1 mg-60 mg of MNA per 100 total grams.

22. The wakame extract of claim 19, wherein the extract is enriched with 60.1 mg-75 mg of MNA per 100 total grams.

23. The wakame extract of claim 19, wherein the extract is enriched with 75.1 mg-90 mg of MNA per 100 total grams.

24. The wakame extract of claim 19, wherein the extract is enriched with 90.1 mg-99 mg of MNA per 100 total grams.

25. A topical composition comprising a wakame extract enriched with MNA.
